(19) United States
(12) Patent Application Publication
(10) Pub. No.: US 2026/0000740 A1
(43) Pub. Date: Jan. 1, 2026

(54) USE OF A L-ASPARAGINASE IN COMBINATION WITH A FERROPTOSIS INDUCER FOR THE TREATMENT OF EXTRANODAL NATURAL KILLER/T-CELL LYMPHOMA

(71) Applicants: Institut National de la Santé et de la Recherche Médicale, PARIS (FR); UNIVERSITÉ PARIS CITÉ, Paris (FR); CENTRE NATIONAL DE LA RECHERCHE SCIENTIFIQUE, Paris (FR); ASSISTANCE PUBLIQUE-HÔPITAUX DE PARIS (APHP), Paris (FR); FONDATION IMAGINE, PARIS (FR)

(72) Inventors: Olivier HERMINE, PARIS (FR); Lucile COURONNE; Amira MAROUF, PARIS (FR); Vahid ASNAFI, PARIS (FR); Guillaume ANDRIEU, PARIS (FR); Mathieu SIMONIN, PARIS (FR)

(21) Appl. No.: 18/880,694

(22) PCT Filed: Jul. 3, 2023
(86) PCT No.: PCT/EP2023/068266

§ 371 Date: Jan. 2, 2025

(30) Foreign Application Priority Data
Jul. 4, 2022 (EP) 22305993.2

(51) Int. Cl. A61K 38/50; A61K 31/439; A61P 35/00

(52) U.S. Cl. CPC A61K 38/50; A61K 31/439; A61P 35/00; C12Y 305/01001

(57) ABSTRACT

Extranodal natural killer/T-cell lymphoma (ENKTCL) is an aggressive haematological malignancy. The treatment of ENKTCL is dependent on the extent of the tumor. However the use of L-asparaginase-containing regimens obtained impressive outcomes as induction or salvage treatment for ENKTCL. Although more than 70% of early-stage patients can achieve long-term survival, patients with advanced-stage disease had extremely poor prognosis even after asparaginase-based chemotherapy regimens. There is thus a medical need for improving the treatment of ENKTCL with L-asparaginase. Now the inventors demonstrate the interest of the use of L-asparaginase in combination with a ferroptosis inducer for the treatment of ENKTCL. In particular, combination of APR-246 and Erwinase R has synergistic effects in KHYG-1 cells. The present invention thus relates to the use of a L-asparaginase in combination with a ferroptosis inducer for the treatment of ENKTCL.

### FIELD OF THE INVENTION

The present invention is in the field of medicine, in particular oncology.

### BACKGROUND OF THE INVENTION

Extranodal natural killer/T-cell lymphoma (ENKTCL) is an aggressive haematological malignancy and accounts for 5.2% and 3% of all non-Hodgkin lymphoma in the Far East and Central/South America, respectively. This neoplasm is indeed more prevalent in regions of Asia and Latin America and most commonly involves the sinonasal tract, presenting with signs of nasal obstruction, epistaxis, or sinus infection. It is a locally destructive and angioinvasive neoplasm. The treatment of ENKTCL is dependent on the extent of the tumor. However the use of L-asparaginase-containing regimens obtained impressive outcomes as induction or salvage treatment for ENKTCL, yielding a CR rate of 46-50% in patients with refractory ENKTCL (A. Jaccard, B. Petit, S. Girault, F. Suarez, R. Gressin, J.-M. Zini, et al. L. -Asparaginase-based treatment of 15 western patients with extranodal NK T-cell lymphoma and leukemia and a review of the literature Ann Oncol., 20 (1) (2008 Jul. 22), pp. 110-116). Although more than 70% of early-stage patients can achieve long-term survival, patients with advanced-stage disease had extremely poor prognosis even after asparaginase-based chemotherapy regimens. There is thus a medical need for improving the treatment of ENKTCL with L-asparaginase.

### SUMMARY OF THE INVENTION

The present invention is defined by the claims. In particular, the present invention relates to the use of a L-asparaginase in combination with a ferroptosis inducer for the treatment of Extranodal natural killer/T-cell lymphoma (ENKTCL).

### DETAILED DESCRIPTION OF THE INVENTION

The first object of the present invention relates to a method of treating ENKTCL in a patient in need thereof comprising administering to the patient a therapeutically effective combination of a L-asparaginase with a ferroptosis inducer, wherein administration of the combination results in enhanced therapeutic efficacy relative to the administration of the L-asparaginase alone.

As used herein, the term “ENKTCL” has its general meaning in the art and refers to the Extranodal natural killer/T-cell lymphoma. ENKTCL is an aggressive malignancy of putative NK-cell origin, with a minority deriving from the T-cell lineage.

As used herein, the term “treatment” or “treat” refers to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A further object of the present invention relates to a method for enhancing the therapeutic efficacy of L-asparaginase administered to a patient as part of a treatment regimen for ENKTCL, the method comprising administering a pharmaceutically effective amount of a ferroptosis inducer to a patient in combination with the L-asparaginase.

As used herein, the expression “enhancing therapeutic efficacy,” relative to cancer refers to a slowing or diminution of the growth of cancer cells, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, a decrease of immune-adverse effects or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy. As used herein, the expression “relative to” when used in the context of comparing the activity and/or efficacy of a combination composition comprising the L-asparaginase with the ferroptosis inducer to the activity and/or efficacy of the L-asparaginase alone, refers to a comparison using amounts known to be comparable according to one of skill in the art.

A further object of the present invention relates to a method of preventing relapse in patient suffering from ENKTCL after a treatment with a L-asparaginase comprising administering to the patient a therapeutically effective amount of a L-asparaginase in combination with a ferroptosis inducer.

As used herein, the term “relapse” refers to reappearance of the cancer after an initial period of responsiveness (e.g., complete response or partial response). The initial period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%. More generally, a response (e.g., complete response or partial response) can involve the absence of detectable MRD (minimal residual disease). In some embodiments, the initial period of responsiveness lasts at least 1, 2, 3, 4, 6, 8, 10, or 12 months; or at least 1, 2, 3, 4, or 5 years.

A further object of the present invention relates to a method of preventing resistance to L-asparaginase therapy comprising administering to the patient a therapeutically effective amount of a L-asparaginase in combination with a ferroptosis inducer.

As used herein the term “resistance to L-asparaginase therapy” is used in its broadest context to refer to the reduced effectiveness of L-asparaginase to inhibit the growth of a cell, kill a cell or inhibit one or more cellular functions, and to the ability of a cell to survive exposure to an L-asparaginase to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

As used herein, the term “L-asparaginase” or “L-ASP” has its general meaning in the art and refers to refers to any enzyme that catalyses the hydrolysis of asparagine to aspartic acid. For example, it includes bacterial forms of L-ASP. In particular, L-ASP from E. coli was the first enzyme drug used as L-ASP and has been marketed as Elspar® in the USA or as Kidrolase® and L-asparaginase Medac® in Europe. L-ASP have also been isolated from other microorganisms, e.g., an L-ASP protein from Erwinia chrysanthemi, named crisantaspase that has been marketed as Erwinase®. Modified L-ASP have also been developed such as methoxy-polyethyleneglycol (mPEG) so-called pegaspargase, marketed as Oncaspar® (Enzon Inc., USA) or PEGylated L-ASP from Erwinia as described in the International Patent Application WO 2011/003886.

In some embodiments, the L-asparaginase of the present invention is Erwinase® having the amino acid sequence as set forth in SEQ ID NO:1.

>Protein sequence for asparaginase (Erwinia
chrysanthemi) monomer
SEQ ID NO: 1
ADKLPNIVILATGGTIAGSAATGTQTTGYKAGALGVDTLINAVPEVKKL
ANVKGEQFSNMASENMTGDVVLKLSQRVNELLARDDVDGVVITHGTDTV
EESAYFLHLTVKSDKPVVFVAAMRPATAISADGPMNLLEAVRVAGDKQS
RGRGVMVVLNDRIGSARYITKTNASTLDTFKANEEGYLGVIIGNRIYYQ
NRIDKLHTTRSVFDVRGLTSLPKVDILYGYQDDPEYLYDAAIQHGVKGI
VYAGMGAGSVSVRGIAGMRKAMEKGVVVIRSTRTGNGIVPPDEELPGLV
SDSLNPAHARILLMLALTRTSDPKVIQEYFHTY

As used herein, the term “ferroptosis” denotes a regulated cell death program iron dependent characterized by the accumulation of lipid peroxides. Ferroptosis is initiated by elevated mitochondrial ROS production and failure in the oxidative stress response mediated by the GSH redox system, leading to the production of toxic peroxidised lipids (Dixon S. J. et al., Cell. 2013).

As used herein, the term “ferroptosis inducer” denotes a compound able to increase ferroptosis occurrence. Ferroptosis inducers are well-known in the art. As example, the ferroptosis inducer may be APR-246, Ras Synthetic Lethal 3 (RSL3), ML162, ML210, acrolein, erastin, Imidazole Ketone Erastin (IKE), Piperazine Erastin (PE), sulfasalazine, sorafenib, Ferroptosis Inducer 56 (FIN56), Ferroptosis inducer endoperoxide (FIN02), Caspase-Independent Lethal 56 (CIL56), mevalonate-derived coenzyme Q10, buthionine sulfoximine (BSO), amentoflavone, dihydroartemisinin (DHA), typhaneoside, artesunate, Withaferin A (WA), auranofin.

In some embodiments, the ferroptosis inducer is APR-246. APR-246, also named Eprenetapopt or PRIMA-1MET (CAS number: 5291-32-7) is a small organic molecule of formula (I). APR-246 is known to exerts a p53-independent function via the depletion of glutathione (GSH) and the accumulation of mitochondrial ROS, leading to the induction of ferroptosis. The IUPAC of APR-246 is 2-(hydroxymethyl)-2-(methoxymethyl)-1-azabicyclo[2.2.2]octan-3-one

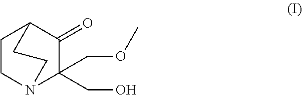

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is the L-asparaginase and wherein the other drug is a ferroptosis inducer. In some instance, the combination of the present invention results in the synthetic lethality of the cancer cells.

In particular, the combination of the L-asparaginase with the ferroptosis inducer results in synergistic effects as demonstrated in the EXAMPLE. As used herein, the term “synergistic effect” to action of two therapeutic agents such as, for example, (a) a L-asparaginase, and (b) an a ferroptosis inducer, producing an effect, for example, reducing the progression of the cancer, which is greater than the simple addition of the effects of each drug administered by themselves. A synergistic effect can be calculated using suitable methods such as described in the EXAMPLE. In particular, the Sigmoid-Emax equation (Holford, N. H. G. and Scheiner, L. B., Clin. Pharmacokinet. 6:429-453 (1981)), the equation of Loewe additivity (Loewe, S. and Muischnek, H., Arch. Exp. Pathol Pharmacol. 114:313-326 (1926)) and the median-effect equation (Chou, T. C. and Talalay, P., Adv. Enzyme Regul. 22:27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Synergy may be further shown by calculating the synergy score of the combination according to methods known by one of ordinary skill. The synergy can be assessed with the MacSynergy II (Prichard, M. N. and C. Shipman, Jr. 1990. A three-dimensional model to analyze drug-drug interactions. Antiviral Res. 14:181-206). This program allows the three-dimensional examination of drug interactions of all data points generated from the checkerboard combination of two inhibitors with Bliss-Independence model. Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB=faA+faB−faA·faB with faA and faB representing the fraction of possible responses, e.g. % inhibition, of compounds A and B at amounts dA and dB, respectively, and describes the % inhibition of a combination of compounds A and B at amount (dA+dB). If faAB>faA+faB−faA·faB then we have Bliss synergy; if faAB<faA+faB−faA·faB then we have Bliss antagonism. The 95% synergy/antagonism volumes are the summation of the differences between the observed inhibition and the 95% confidence limit on the prediction of faAB under the Bliss independence model.

As used herein, the expression “therapeutically effective amount” is meant a sufficient amount of the active ingredient (e.g. ferroptosis inducer) for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the drug of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1. Combination of APR-246 and Erwinase® has synergistic effects in KHYG-1 cells.

A—Cell viability quantification of KHYG-1 cells exposed for 72 hours to APR-246 20 uM, Kidrolase 0.5 UI/mL or Erwinase 0.5 UI/mL, as single agent or in combination as indicated. Viable cells are defined as Annexin V-/PI-cells. Results are represented as the mean percentage±SD of two technical replicates and are representative of 4 independent experiments.

B—Viability curves of KHYG-1 cells exposed for 72 hours to a dose range of L-Asparaginases, as single agent or in combination with APR-246 20 uM. Viable cells are defined as Annexin V-/PI-cells. Viability curves were obtained using linear regression analysis. Results are represented as the mean percentage±SD of two technical replicates and are representative of 4 independent experiments.

C—Illustrative synergy map of 72 h co-treatment of KHYG-1 cells with APR-246 and Erwinase.

FIG. 2. APR-246 targets metabolic cell vulnerabilities induced by Erwinase to promote ferroptosis. A and B.

### EXAMPLE

### Methods

### Cell Lines and Culture Conditions

NALM6 human acute lymphoblastic leukemia cell line was gently provided by Institut Curie. The human natural killer (NK) leukemia/lymphoma cell lines KHYG-1 (ACC 725-RRID: CVCL_2976), YT1 (RRID: CVCL_EJ05) and MEC04 cell lines were respectively purchased from Leibniz Institute DSMZ-German Collection of Microorganisms and Cell Cultures GmbH, provided kindly as a gift from Dr. Olivier Bernard (Gustave Roussy Institute), and also gently provided by Prof Philippe Gaulard. 15

All these cell lines were cultured in RPMI-1640 media containing 10% FBS, 1% Penicillin-streptomycin and 1% Glutamine. Human IL2 purchased from Miltenyi Biotec (human IL2 “improved sequence”, premium grade, reference 130-097-746) was freshly added into the media at the concentration of 100 UI/mL (=10 ng/mL) for KHYG1 and MEC04 culture. All the cells were cultured in a humidified atmosphere of 95% air and 5% CO2 at 37° C. and routinely tested for mycoplasma infection.

### Drugs and Reagents

APR-246 was purchased from Sigma-Aldrich (reference SML1789). Unused L-Asparaginases from E. Coli (Kidrolase®) and from Erwinia chrysanthemi (Erwinase®) were kindly provided by the Pharmacy department of Cochin Hospital.

### Cell Viability Measurement by Flow Cytometry

KHYG-1 cells were seeded into flat wells of a 96-wells plate (Falcon) at a final concentration of 0.1 10\hat{ }6 cells/well. APR-246 was dispensed at a final concentration of 20 uM and Asparaginases were dispensed at a final concentration range from 0.1 to 1 UI/mL. Plates were incubated at 37° C. and 5% CO2 for 72 hours in a humidity chamber. Cell viability was determined by flow cytometry using Annexin V (APC Annexin V, BD Biosciences, ref: 550474, 1:100 dilution)/Propidium Iodide (PI) (Propidium Iodide, Invitrogen, ref: BMS500FI-100, 1:20 dilution) staining. After 20 min incubation at 4° C. away from light, flow cytometry was performed using BD Fortessa cytometer (with HTS device for plate reading).

### Data Analysis and Statistics

Flow cytometry data were analysed using FlowJo software and GraphPad-Prism 9. R software and Rstudio interface (version 1.4.1106) were used for synergy score calculation. Calculations were based on the Loewe model.

### Results

### Asparaginase Resistance Increases Mitochondrial Oxidative Stress Levels and Enhances Glutaminolysis.

We first determined Asparaginase sensitivity in our different cell lines. We observed that MEC-04 and YT1 cell lines showed high sensitivity to both Kidrolase and Erwinase whereas NALM6 and KHYG1 were resistant.

Given that ASNase-R induction has been shown to be associated with epigenetic silencing of ASNs promoter⁹, we assessed ASNs expression and ASNs promoter methylation in our cell lines both at baseline and over ASNase treatment (Data not shown). We found that ASNase-R cell lines were associated with increased ASNs expression and low levels of promoter methylation at baseline. In contrast, we observed ASNs hypermethylation status and low level of ASNs expression in ASNase-S cell lines in the same conditions (Data not shown). Upon ASNase depletion, ASNs expression was inducible only in ASNase-R cells, which is consistent with ASNs promoter hypomethylation and previous reports.

Then, we wanted to uncover the metabolic changes induced by ASNase resistance. By performing metabolomics analysis in pretreated cells, we showed that metabolic reprogramming occurs in cells able to resist to ASN depletion. Compared to untreated condition, KHYG1 treated cells exhibits enhanced glutaminolysis and decreased GSH levels (Data not shown) resulting in reduced GSH to oxidized GSSG ratio which is an indicator of cellular redox inbalance (Data not shown).¹⁰ Indeed, glutathione (GSH) is the most abundant non-enzymatic antioxidant molecule in the cell and is critical for cell survival and redox homeostasis, acting as a free radical scavenger and inhibitor of lipid peroxidation.¹¹ Therefore, we performed functional validation by specific quantification of GSH and mitochondrial ROS levels in tumor cells treated or not with ASNase and confirmed that ASNase resistance results in mitochondrial ROS accumulation (Data not shown) and GSH (Data not shown) reduction leading to mitochondrial oxidative stress and antioxidant defense impairment.

Interestingly, treatment with Erwinase induces higher ROS levels and GSH decrease compared to Kidrolase suggesting increased oxidative stress and redox imbalance upon Erwinase. (Data not shown).

Taken together, these data suggest that ASNase resistance in our models arises from favorable epigenetic and metabolic contexts, allowing de novo synthesis of Asn and energetic adaptation including harmful metabolic reprogramming in response to ASNase depletion. These metabolic changes results in tumor cell vulnerability which is an interesting target to harness. In particular, our results suggest the use of therapeutic agents able to worsen GSH/ROS imbalance inducing further accumulation of ROS and cell death.

### Synergistic Efficacy of APR and ASNase Combination In Vitro and In Vivo.

ASNase is the standard of care in ALL and ENKTCL. Despite its efficacy, 40% of the patients experienced refractory disease after frontline therapy. After having evaluated the efficacy of ASNase in three ENKTCL and one B-ALL cell lines allowing us to identify ASNase-R cell lines, we investigated whether ASNase sensitivity could be restored by combination with APR-246.

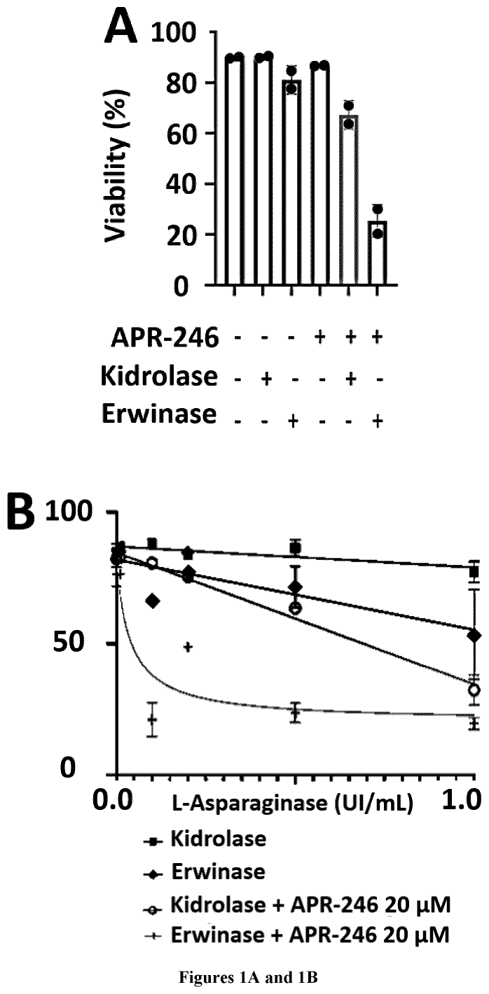

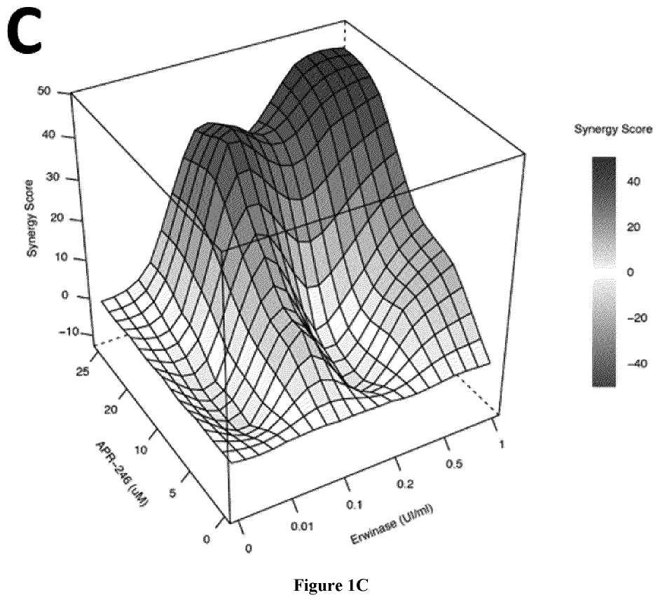

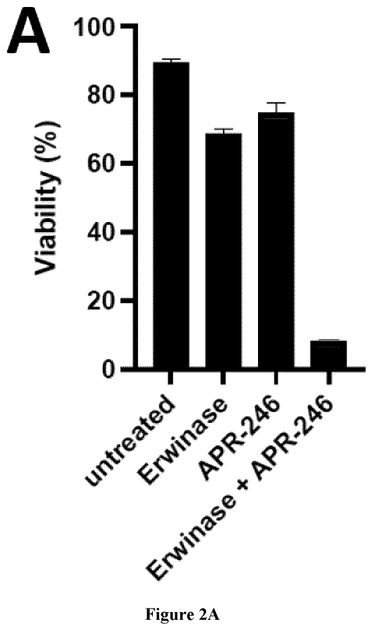

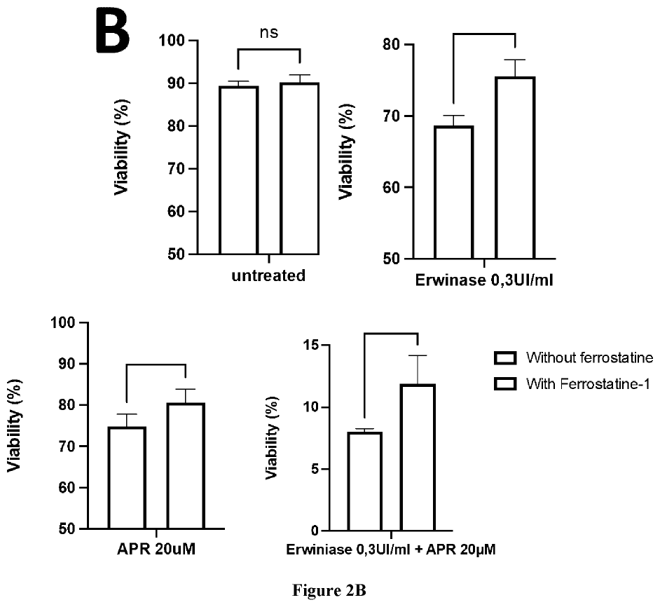

The addition of APR 20 uM (Data not shown) to increasing doses of ASNase induced significant cytotoxicity compared to each treatment on its own. Strikingly, we observed a synergistic effect of APR-246 with Erwinase confirmed at different concentrations of each drug by calculating the Z-score, but not with Kidrolase and APR-246 combination (FIGS. 1A to 1C). These results confirm the synergistic efficacy of APR and ASNase combination in vitro and in vivo.

### The Combination of APR-246 and Erwinase Synergistically Promotes Ferroptosis by Targeting Metabolic Cell Vulnerabilities Due to Erwinase Induced Glutamine Depletion.

### Erwinase Promotes Cell Sensitivity to APR-246 Through Glutamine Depletion.

To decipher the mechanisms underlying the greatest synergistic effect observed by combining APR-246 to Erwinase, we performed metabolomic analysis in tumor cells and in the supernatant after one day treatment with Kidrolase or Erwinase.

While both induce decrease of GSH and GSH/GSSG ratio resulting in GSH/ROS unbalance, the effect was more pronounced with Erwinase (Data not shown). Glutamine (Gln) intake from extracellular compartment is converted in Glutamate (Glu) by glutaminase (GLS) and participates to cancer cells protection from oxidative stress through solute carrier family 7-member 11 (SLC7A11) expression. SLC7A11 encodes the cystine/Glu antiporter xCT which imports cysteine into the cell in exchange for Glu uptake. Thus, in our models, we assessed Gln extracellular levels in the supernatant of cells cultured with each drug and observed that Erwinase induces deeper extracellular Gln depletion compared to Kidrolase (Data not shown).

To confirm that Kidrolase and Erwinase differential effects relies on Gln intake, we performed Gln addition combined to Erwinase treatment and observed that Gln rescue partially prevents cell death. This result shows that Erwinase-induced Gln depletion participates to direct cytotoxicity whereas no effect was observed by adding Gln to other treatments alone (Kidrolase or APR-246) (Data not shown). Also, extracellular Gln addition significantly rescues intracellular GSH levels upon Erwinase treatment (Data not shown), suggesting that Erwinase's glutaminase activity promotes metabolic vulnerability leading to enhanced APR-246 susceptibility.

### Glutamine/Glutathione Metabolism: An Achille Heel's of ASNase-R Cells

xCT is overexpressed in many cancers and contributes to tumor progression and chemoresistance by participating to GSH biosynthesis, allowing the maintenance of intracellular GSH reserves, thus protecting against excessive ROS accumulation.^{6,12}

Knowing that SLC7A11 is a determinant of APR-246 response¹³, we assessed SLC7A11 mRNA expression in our models. We observed that SLC7A11 low expression is predictive of APR-246 sensitivity in our cell lines (Data not shown), suggesting that impaired Gln intake may impacts APR-246 mediated cell death.

Indeed, more than promoting GSH depletion by direct binding, APR-246 has also been described as an indirect modulator of xCT expression, suggesting that APR-246 may synergize with drugs inducing GSH/ROS unbalance to induce cancer cell death.

Altogether, our results suggest that extracellular depletion of both Asn and Gln using Erwinase leads to increased mitochondrial oxidative stress and impaired Gln uptake reducing GSH levels.

### APR-246 Targets Metabolic Cell Vulnerabilities Induced by Erwinase to Promote Ferroptosis

APR-246 is converted to the Michael acceptor methylene quinuclidinone (MQ) which inhibits activity of the antioxidant metabolite GSH.¹⁴ Therefore, as we previously showed that Erwinase decreases on its own GSH levels, we evaluated whether the lethal effects of APR-246 and Erwinase combination in ASNase-R cells were due to deeper GSH inhibition. APR-246 combined to Erwinase dramatically decreased GSH levels in our models (Data not shown).

Covalent binding of MQ decreased the level of GSH, thereby shifting the intracellular balance between ROS generation and antioxidation toward an increase in ROS levels. ROS accumulation induces lipid peroxidation, which is harmful for tumor cells in particular if GSH levels are low. Indeed, GSH acts a lipid peroxidation suppressor via GPX4 enzyme.

Thus, we next examined lipid peroxidation in KHYG1 cell line upon APR-246+Erwinase and showed a significant increase in lipid peroxidation levels (Data not shown) consistent with treatment-induced cell death (FIG. 2A).

Given that accumulation of lipid peroxides leads to ferroptosis which is an iron-dependent mechanism of cell death, our results strongly suggest that APR-246+Erwinase promote ferroptosis in our model.

In addition, the GSH decrease and lipid peroxidation accumulation observed in APR-246+Erwinase treated cells was reproduced using the ferroptosis inducer RSL3 combined with Erwinase and reversed by the addition of ferrostatin-1 which is a ferroptosis inhibitor (FIG. 2B). Consistently, the decrease in cell viability was also observed in RSL3+Erwinase-treated cells, while abrogated by co-treatment with ferrostatin-1 (Data not shown).

These results strengthen that inhibition of GSH in ASNase-R cancer cells with APR-246 and Erwinase leads to higher ROS levels by disrupting the balance between ROS generation and antioxidation dependent on Gln/GSH metabolism, resulting in cell death by ferroptosis (Data not shown).

The results demonstrate the interest of the use of L-asparaginase in combination with a ferroptosis inducer for the treatment of ENKTCL.

### REFERENCES

Throughout this application, various references describe the state of the art to which this invention pertains. The disclosures of these references are hereby incorporated by reference into the present disclosure.

1. A method of treating Extranodal natural killer/T-cell lymphoma (ENKTCL) and/or preventing resistance to L-asparaginase therapy in a patient in need thereof comprising administering to the patient a therapeutically effective combination of an L-asparaginase with a ferroptosis inducer.

2. A method for enhancing the therapeutic efficacy of L-asparaginase administered to a patient as part of a treatment regimen for ENKTCL, the method comprising administering a pharmaceutically effective amount of a ferroptosis inducer to the patient in combination with the L-asparaginase, wherein administration of the combination results in enhanced therapeutic efficacy relative to the administration of the L-asparaginase alone.

3. A method of preventing relapse in patient suffering from ENKTCL after a treatment with a L-asparaginase comprising administering to the patient a therapeutically effective amount of a L-asparaginase in combination with a ferroptosis inducer.

4. (canceled)

5. The method according to claim 1, wherein the L-asparaginase has the amino acid sequence as set forth in SEQ ID NO:1.

6. The method according to claim 1, wherein the ferroptosis inducer is APR-246.

7. The method according to claim 2, wherein the L-asparaginase has the amino acid sequence as set forth in SEQ ID NO:1.

8. The method according to claim 2, wherein the ferroptosis inducer is APR-246.

9. The method according to claim 3, wherein the L-asparaginase has the amino acid sequence as set forth in SEQ ID NO: 1.

10. The method according to claim 3, wherein the ferroptosis inducer is APR-246.


SEQUENCE LISTING (1 sequence)

SEQ ID NO: 1 - protein, 327 aa, Erwinia chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY